(19) United States
(12) Patent Application Publication
(10) Pub. No.: US 2004/0024415 A1
(43) Pub. Date: Feb. 5, 2004

(54) Flanged prosthesis with radial and peripheral seam

(76) Inventor: Eric Perouse, Paris (FR)

Correspondence Address: WENDEROTH, LIND & PONACK, L.L.P., 2033 K STREET N. W., SUITE 800, WASHINGTON, DC, 20006-1021, US

(21) Appl. No.: 10/443,020

(22) Filed: May 22, 2003

(30) Foreign Application Priority Data
May 24, 2002 (FR) 02 06387

(51) Int. Cl. A61F 2/06

(52) U.S. Cl. 606/153; 623/1.36

(57) ABSTRACT

The flanged prosthesis comprises a tubular body equipped at one end with an external flange designed for anastomosis of the prosthesis to a tubular duct equipped with an opening, in which prosthesis said flange is made of a textile material and comprises at least one seam (324, 426) shaping it at the end of the tubular body. Said flange comprises a first ring which is integral, along its inner contour, with the end of the tubular body, and a second ring (24) surrounding said tubular body and extending substantially against the first ring (20), these rings being connected to one another along their outer contours. The first ring is made in one piece with the tubular body, and the first and second rings are connected to one another by an external peripheral seam (426).

The present invention relates to a flanged prosthesis of the type comprising a tubular body equipped at one end with an external flange designed for anastomosis of the prosthesis to a tubular duct equipped with an opening, in which prosthesis said flange is made of a textile material and comprises at least one seam shaping it at the end of the tubular body, said flange comprising a first ring which is integral, along its inner contour, with the end of the tubular body, and a second ring surrounding said tubular body and extending substantially against the first ring, these rings being connected to one another along their outer contours.

It also relates to a method for manufacturing such a flanged prosthesis.

Flanged prostheses of this type are used as vascular prostheses, in vascular surgery, for treating diseased blood vessels, particularly portions of arteries, by bypass or by replacement.

As is illustrated in document FR-A-2,751,867 describing a prosthesis of this kind, these prostheses are connected to a tubular duct in the body, for example a vein or an artery, facing an opening formed in this duct.

The flange, which is provided at the end of the tubular body, allows the prosthesis to be attached using plastically or elastically deformable clips or staples.

The flanged prosthesis described in document FR-A-2,751,867 is made either by thermoforming its end portion or alternatively by bonding or welding of a separate flange attached to the end of the tubular body.

Said document deals only with the technologies that can be employed, without giving a precise description of the actual methods of manufacturing this flanged prosthesis.

Thus, it is very difficult, using these technologies, to produce in a simple way a flanged prosthesis whose structure is satisfactory and which can be implanted reliably in the human body.

Other solutions are proposed in patent application 2,799,362.

The object of the invention is to make available a flanged prosthesis which can be manufactured on an industrial scale and which has satisfactory reliability for durable implantation in the human body, and a method for manufacturing it.

To this end, the subject of the invention is a flanged prosthesis of the aforementioned type, characterized in that the first ring is made in one piece with the tubular body, and the first and second rings are connected to one another by an external peripheral seam.

According to particular embodiments, the prosthesis comprises one or more of the following features:

the first ring comprises at least one transverse seam;

the transverse seam or each transverse seam extends substantially radially; and

the first ring comprises a single transverse seam.

A further subject of the invention is a method for manufacturing a flanged prosthesis of the type comprising a tubular body equipped at one end with an external flange designed for anastomosis of the prosthesis to a tubular duct equipped with an opening, said method comprising the following steps:

manufacturing a prosthesis blank made of textile material and comprising a tubular skirt from one end of which at least one tubular arm intended to form the tubular body of the prosthesis emerges, the diameter of the tubular skirt being greater than the diameter of the arm,

cutting the tubular skirt around and at a distance from the tubular arm in order to form a first ring continuing the tubular body,

manufacturing a substantially flat annular element from a textile material, said element having an outer contour substantially identical to that of the first ring; and

sewing said annular element to said first ring using a peripheral seam formed along their outer contours.

According to particular embodiments, the method comprises one or more of the following features:

for formation of the first ring, it comprises a step in which the tubular skirt is folded about a rigid dummy comprising at least one disk-shaped part around which the flange is made; and

the step in which the blank is manufactured comprises:

a step of manufacturing a first blank comprising a tubular skirt, from one end of which a first tubular arm and a second tubular arm emerge, the first arm being intended to form the tubular body of the prosthesis;

a step in which the second tubular arm is cut at the level of the tubular skirt;

a step in which the orifice obtained by cutting the second arm is closed off by a seam.

The invention will be better understood from reading the description which follows, which is given solely by way of example and in which reference is made to the drawings, where:

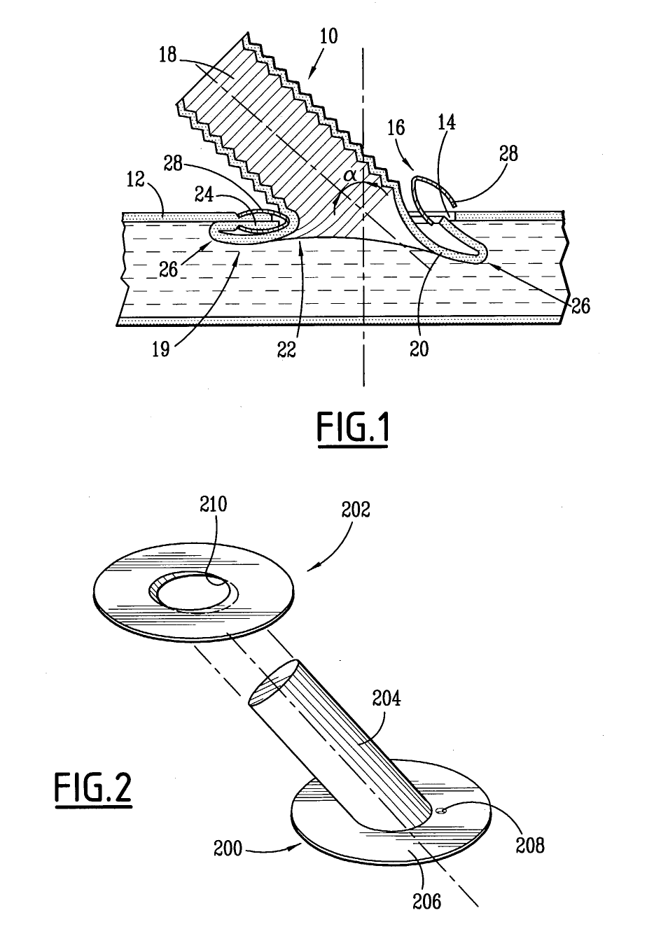

FIG. 1 is a view in longitudinal section of a flanged prosthesis in the process of being implanted;

FIG. 2 is a perspective view of a tool for manufacturing a flanged prosthesis according to the invention;

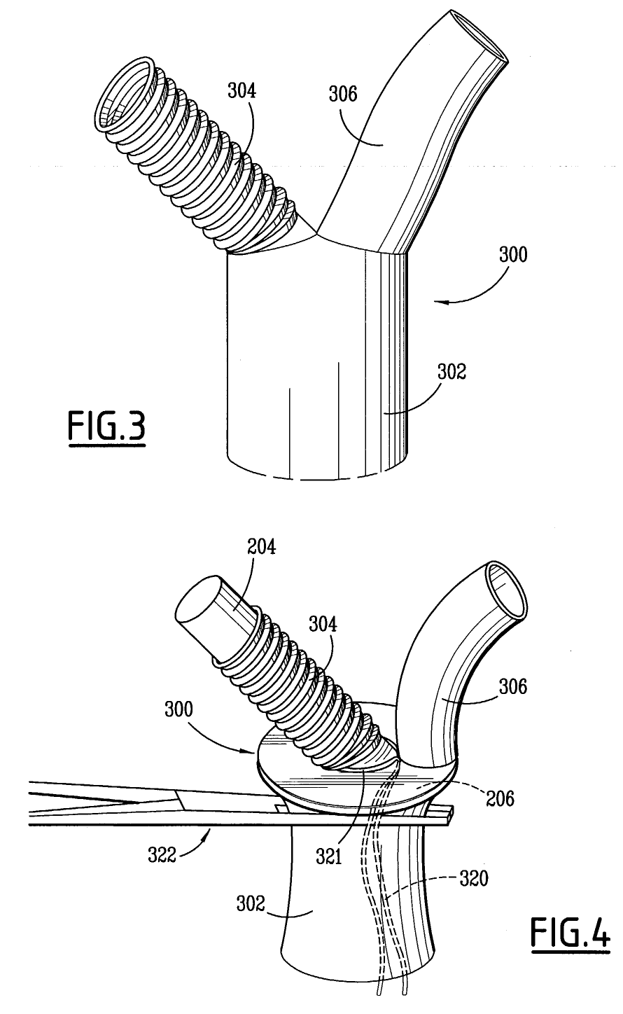

FIG. 3 is a perspective view of a vascular prosthesis blank used for manufacture of the flanged prosthesis;

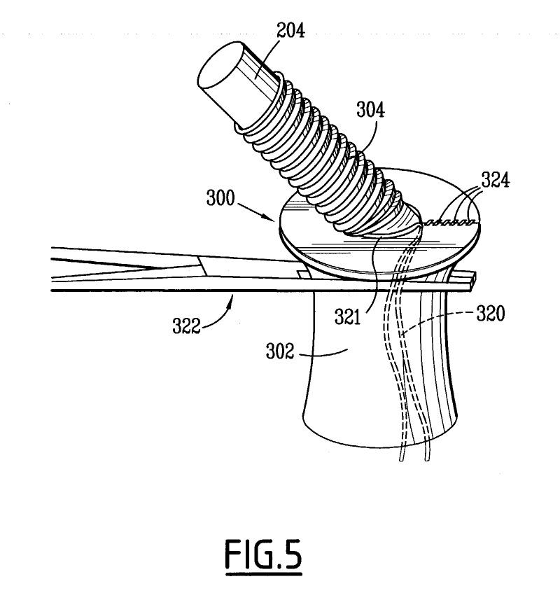

FIGS. 4 and 5 are perspective side views illustrating two successive steps in the method of manufacturing the prosthesis;

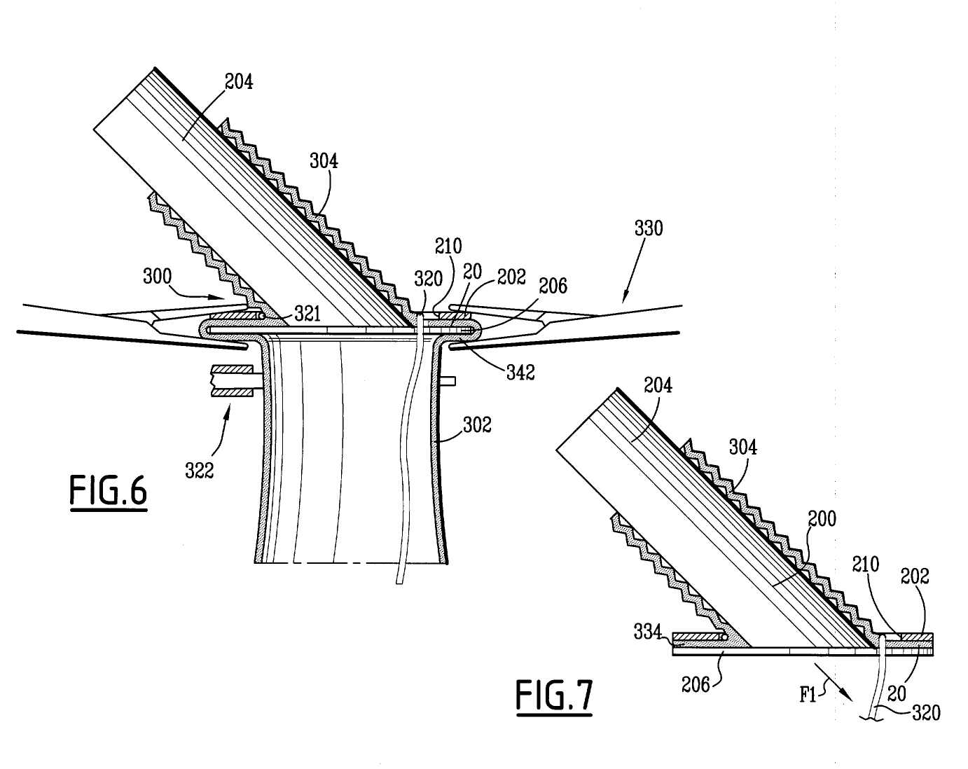

FIGS. 6 and 7 are side views in cross section illustrating the next steps in the method of manufacturing the prosthesis;

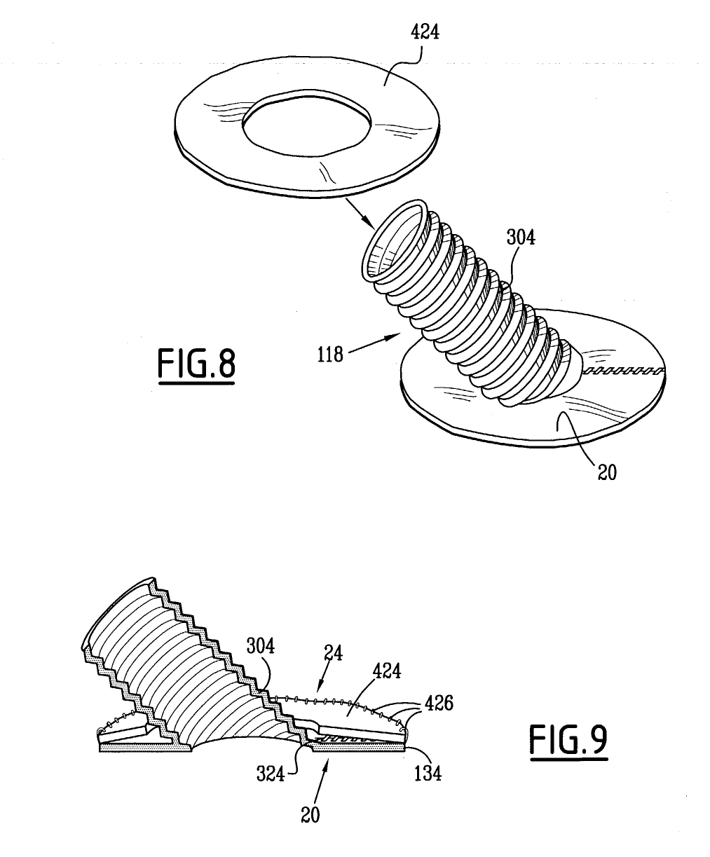

FIG. 8 is a perspective view of the flanged prosthesis according to the invention during its manufacture; and

FIG. 9 is a perspective and cross-sectional view of the assembled flanged prosthesis.

FIG. 1 generally depicts a flanged prosthesis 10 in the process of being implanted on a tubular duct 12 such as an artery. This duct is equipped, in its side wall, with an opening 14, to the periphery of which the prosthesis 10 is connected by fixing means 16.

The prosthesis 10 comprises a flexible tubular body 18 and an end flange 19.

The body 18 consists of a ringed tubular duct, that is to say one which has a permanent helically-shaped deformation along most of its length. This type of deformation is also known as “ribbing”.

At its connection end bearing the flange 19, the tubular body 18 is continued by a first ring 20. The axis of the first ring 20 defines, with the axis of the tubular body 18, an angle a substantially equal to 45°.

The body 18 and the first ring 20 are connected to one another along the inner contour, labeled 22, of the first ring.

A second ring 24 continues the first ring 20. The latter is applied substantially on the face of the first ring 20 continuing the outer surface of the body 18. It thus surrounds the end of the body 18.

The first and second rings are connected along their outer contour 26 and together form the flange 19.

As is illustrated in FIG. 1, the means 16 for joining the flanged prosthesis to the tubular duct 12 comprise a set of clips 28 which are engaged through the opening 14 and bear, on the one hand, on the outer surface of the tubular duct 12 and, on the other hand, on the second ring 24, thus pressing this second ring flat against the inner surface of the tubular duct 12.

FIG. 2 shows a tool for manufacturing a flanged prosthesis according to the invention.

This tool comprises a tool body 200 and a washer 202. These two elements are made of metal.

The tool body comprises a straight rod 204, to one end of which an elliptical disk 206 is attached. The end of the rod 204 receiving the disk is beveled at 45°, so that the axis of the disk 206 defines, with the axis of the rod 204, an angle of close to 45°. The axis of the rod 204 passes substantially through the center of the elliptical disk 206. The axis of the rod 204 is projected orthogonally onto the disk 206 along the main axis of the disk.

An open hole 208 passes through the disk 206. This hole is formed at the base of the straight rod 204 along the main axis of the ellipse on the side where the rod 204 defines, with the disk 206, a maximum angle of 135°.

The washer 202 has an overall elliptical shape. Its outer contour corresponds to the contour of the elliptical disk 206.

Passing through the center of the washer is an elliptical aperture 210 whose cross section is slightly greater than the cross section of the straight rod 204 taken on a plane parallel to the disk 206. The side wall of the aperture 210, defined within the thickness of the washer 202, is beveled as a cylindrical envelope in order to correspond to the inclination of the rod 204.

Thus, the rod 204 can be introduced into the aperture 210, thus allowing the washer 202 to slide along the length of the rod 204, while remaining parallel to the disk 206. In its extreme position, the washer 202 fits exactly on what is the upper surface of the elliptical disk 206.

To produce the prosthesis, a primary blank 300 shown in FIG. 3 is used. This blank consists of a forked vascular prosthesis. This prosthesis is normally used to connect a main section to two bypass sections. It thus generally has the shape of a Y.

This forked prosthesis is produced by knitting according to a method known per se. It comprises a main body defined by a tubular skirt 302 with a diameter of about 3 cm. The body is continued by two adjacent arms 304, 306. These are formed by tubular ducts emerging side by side at one end of the body. Each tubular duct has a diameter substantially equal to half the diameter of the skirt 302.

One of the arms 304 is ringed using a method known per se. By contrast, the skirt 302 and the arm 306 are smooth, these having undergone no prior shaping treatment.

To manufacture the prosthesis, and as is illustrated in FIG. 4, the tool body 200 is first introduced into the blank 300 from the open end of the skirt delimiting the main body. More precisely, the rod 204 of the tool body is engaged through the arm 304, the elliptical disk 206 being placed in the region where the skirt 302 meets the arms 304 and 306. In particular, the end of the arm 306 is applied on the upper face of the elliptical disk 206 in the plane containing the hole 208, that is to say in the region of the disk delimiting the maximum angle with the axis of the rod 204.

The cylindrical wall of the skirt 302 is applied on the upper surface of the elliptical disk 206. To ensure that the skirt 302 is pressed satisfactorily against the upper surface of the disk 206, a thread 320 forming a loop is engaged around the base of the arm 304 supported by the rod 204, the two strands of the thread being introduced into the hole 206. These thus pass through the blank 300 in the region of connection of the arms 304 and 306. The two ends of the thread thus extend inside the skirt 302 and emerge at its open end. Pulling on the two ends of the thread ensures that the skirt 302 comes into intimate contact with the entire upper surface of the disk 206, thereby creating a peripheral fold line 321 at the end of the arm 304.

To ensure that the assembly is held in place, a clamp 322 is placed transversely around the entire width of the skirt 302. It is arranged below the disk 206, thus permitting pulling on that part of the skirt 302 surrounding the disk 206.

In the next step, the arm 306 is cut in the plane of the disk 206 and the two edges thus defined at the end of the skirt 302 are brought together by a hand-stitched seam 324, as is illustrated in FIG. 5. The annular region thus delimited in the skirt 302 on the upper face of the elliptical disk 206 forms the first ring 20 of the prosthesis. The seam 324 extends radially along this ring.

In the next step illustrated in FIG. 6, the washer 202 is engaged around the arm 304 supported by the rod 204. This washer is brought to bear against the first ring 20 resting on the upper surface of the elliptical disk 206. Clamps 330 are fitted in place to press the washer 202 onto the first ring 20. For this purpose, one of the jaws of each clamp presses against the washer 202, while the other jaw presses against the part of the skirt 302 resting against the lower surface of the elliptical disk 206.

While the skirt 302 is thus held on each side of the disk 206, the prosthesis blank is shaped by heating with steam. The prosthesis blank thus supported by the tool is subjected to a flow of steam and is brought to a temperature of 120° C. for 20 minutes. At the end of this treatment, the shape of the prosthesis blank imposed by the tool supporting it is marked, by the action of the heat, on the polyester fibers constituting the knit of the prosthesis.

The extending part of the skirt 302, which forms an excess length, is then cut off substantially along the circumference of the disk 206 using scissors or a thermocutter so as to form a basically circular outer peripheral contour 334, as is illustrated in FIG. 7.

The tool body 200 and the washer 202 are then disengaged from the prosthesis blank.

For this purpose, the thread 320 is pulled out and the tool body 200 is released in the direction of the arrow F1 in FIG. 7.

An annular element 424 is cut out from a piece of knitted material having a yarn of the same kind as that used for the portion 118 and with an analogous stitch. It has a slightly elliptical outer shape.

The annular element 424 has an outer contour identical to the desired outer contour of the flange 19 of the prosthesis. The internal cross section of the annular element 424 intended to form the second ring 24 is slightly greater than the elliptical cross section of the beveled end of the tubular element 304.

The annular element 424 is engaged around the tubular element 304, as is illustrated in FIG. 8, until it comes into contact with the first ring 20.

As is illustrated in FIG. 9, in order to assemble the prosthesis, the outer contour 334 of the first ring 20 is connected to the annular element 424 by a peripheral seam 426 in order to form the second ring 24 of the prosthesis. The annular element 424 is sewn onto the first ring 20 while engaged around the tubular portion 304 forming the body 18.

The prosthesis formed in this way is coated with collagen with a view to its implantation in a living organism.

It will be appreciated that the prosthesis thus produced has satisfactory flexibility and that it is therefore well tolerated by the organism. In addition, producing it by sewing means that it can be made at reasonable cost.

The prostheses described here can be made from textile elements of any suitable nature, whether woven or knitted.

1. A flanged prosthesis (10) of the type comprising a tubular body (18) equipped at one end with an external flange (19) designed for anastomosis of the prosthesis to a tubular duct equipped with an opening, in which prosthesis said flange (19) is made of a textile material and comprises at least one seam (324, 426) shaping it at the end of the tubular body (18), said flange comprising a first ring (20) which is integral, along its inner contour, with the end of the tubular body (18), and a second ring (24) surrounding said tubular body (18) and extending substantially against the first ring (20), these rings being connected to one another along their outer contours, wherein the first ring (20) is made in one piece with the tubular body (18), and the first and second rings (20, 24) are connected to one another by an external peripheral seam (426).

2. The prosthesis as claimed in claim 1, wherein the first ring (20) comprises at least one transverse seam (324).

3. The prosthesis as claimed in claim 2, wherein the transverse seam or each transverse seam (324) extends substantially radially.

4. The prosthesis as claimed in claim 2 or 3, wherein the first ring (20) comprises a single transverse seam (324).

5. A method for manufacturing a flanged prosthesis (10) of the type comprising a tubular body (18) equipped at one end with an external flange (19) designed for anastomosis of the prosthesis to a tubular duct equipped with an opening, which method comprises the following steps:
manufacturing a prosthesis blank (300) made of textile material and comprising a tubular skirt (302) from one end of which at least one tubular arm (304) intended to form the tubular body (18) of the prosthesis emerges, the diameter of the tubular skirt (302) being greater than the diameter of the arm (304),
cutting the tubular skirt (302) around and at a distance from the tubular arm (304) in order to form a first ring (20) continuing the tubular body (18),
manufacturing a substantially flat annular element (120) from a textile material, said element having an outer contour substantially identical to that of the first ring; and
sewing said annular element (120) to said first ring using a peripheral seam (426) formed along their outer contours.

6. The method as claimed in claim 5, comprising, for formation of the first ring (20), a step in which the tubular skirt (302) is folded about a rigid dummy (200) comprising at least one disk-shaped part (206) around which the flange (19) is made.

7. The method as claimed in claim 5 or 6, wherein the step in which the blank (300) is manufactured comprises:
a step of manufacturing a first blank (300) comprising a tubular skirt (302), from one end of which a first tubular arm and a second tubular arm (304, 306) emerge, the first arm (304) being intended to form the tubular body (18) of the prosthesis;
a step in which the second tubular arm (306) is cut at the level of the tubular skirt (302);
a step in which the orifice obtained by cutting the second arm (306) is closed off by a seam (324).
